(19) United States
(12) Patent Application Publication
(10) Pub. No.: US 2008/0269832 A1
(43) Pub. Date: Oct. 30, 2008

(54) Device and method for sleep apnea management using SpO2

(75) Inventors: Thomas Kwok Shing Wong, Hong Kong (HK); Joanne Wai Yee Chung, Hong Kong (HK); Winnie Wing Man Yu, Hong Kong (HK); Mayur Danny Indulal Gohel, Hong Kong (HK); Siu Luen Ng, Hong Kong (HK); Ka Lun Fan, Hong Kong (HK); Ching Ching Cheung, Hong Kong (HK); Yin Ki Lau, Hong Kong (HK); Wai Man Au, Hong Kong (HK)

Correspondence Address: ATTN: Ms. Maquita Wong;The Hong Kong Polytechnic University, 1800 Diagonal Road, Suite 600, Mailbox #119, Alexandria, VA, 22314, US

(73) Assignee: The Hong Kong Polytechnic University, Hong Kong (HK)

(21) Appl. No.: 11/790,501

(22) Filed: Apr. 26, 2007

(51) Int. Cl. A61N 1/36

(52) U.S. Cl. 607/42

(57) ABSTRACT

The present invention relates to an anti-sleep apnea device including two electrodes and a pulse oximeter, adapted to be disposed on the user's wrist for releasing electrical pulses to an acupoint of the user, and a method of monitoring and treating sleep-apnea, whereby monitoring occurs via a pulse oximeter and treatment occurs by providing electrical stimulation to specific acupoint on the user.

### BACKGROUND

There are three main types of sleep apnea, namely obstructive, central, and mixed. Obstructive sleep apnea is the blockage of the airway. Central sleep apnea is the failure to control the airway to breathe. Mixed sleep apnea is the combination of obstructive and central sleep apnea. Some common methods for managing sleep apnea are weight management, continuous Positive Airway Pressure (CPAP) device, oral appliance, surgeries and tongue stimulation.

Weight management may not be an effective approach in the long run. Patients may regain their weight. Also, weight management is not recommended for sleep apnea patients who have craniofacial abnormalities.

CPAP device is regarded as a safe and effective alternative for managing sleep apnea, many patients cannot well tolerate the CPAP device. The CPAP devices produce discomfort such as nasal or mouth dryness, psychological discomfort and facial irritation, and increased the number of tossing and turning events during sleep and masks leaking.

With regard to oral appliances, patients may refuse to use them due to the side effects. Oral appliances cause teeth, gum, tongue and jaw discomfort and initial excessive saliva secretion.

Surgery may also produce side effects such as postoperative pain, nasal regurgitation and voice change after receiving surgery.

Electrical stimulation for tongue muscle training may produce discomfort to the users as the electrodes are implanted/placed over the intra-oral area of the patients. The rate of response using the electrical stimulation is variable. Some previous works used an invasive technique to place the electrodes in contact with the hypoglossal nerves. Anesthesia and sterile technique were used. Although positive results were reported with this procedure in obstructive sleep apnea patients, some researchers mentioned that the electrical stimulations increased the number of arousal events while others claimed that the subjects remained asleep during electrical stimulations.

Acupuncture is a technique of inserting and manipulating needles into “acupuncture points” on the body. Luo-connecting acupuncture points are located at meridian points, i.e. connected points across the body which affect the lungs. The Lung luo-connecting channel follows the Lung channel into the palm. If acupuncture is applied for heat of the chest and back, sweating, and sudden oedema of the four limbs, and shivering and cold of the chest and back, diminished and shortness of breath, it is the luo-connecting points that are contacted. However, sleep apnea has never been indicated to be treated by luo connecting points.

Pulse oximetry focuses on the indirect measurement of the amount of oxygen in a patients blood and changes in blood volume in the skin. Oximetry allows a measurement of a patients respiratory sufficiency. However, whereas a pulse oximeter can measure respiratory insufficiency, treatment is not readily applied, allowing the user, during a sleep period, to continue to undergo apnea.

It is an object of the present invention to overcome the disadvantages and problems in the prior art.

### DESCRIPTION

The present system proposes an anti-sleep apnea device including two electrodes and a pulse oximeter, adapted to be disposed on the user's wrist for releasing electrical pulses to an acupoint of the user.

It is also a purpose of this invention to provide a method of monitoring sleep-apnea and, in the case of the occurrence of such, a treatment method whereby monitoring occurs via a pulse oximeter and treatment occurs by providing electrical stimulation to specific acupoint on the user.

It is further a purpose of this invention to provide a device and methods for treating sleep apnea by detecting and treating in a noninvasive manner in order to avoid harm to the user due to prolonged apnea during sleep.

These and other features, aspects, and advantages of the apparatus and methods of the present invention will become better understood from the following description, appended claims, and accompanying drawings where:

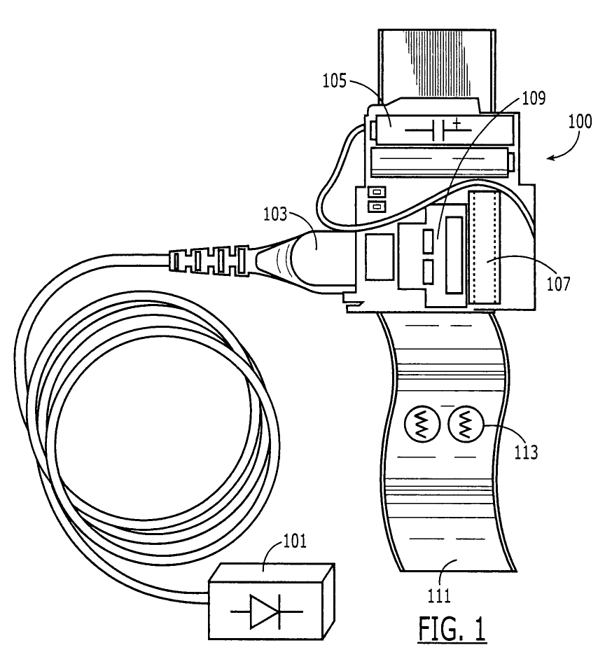

FIG. 1 shows an anti-sleep apnea device of the present invention.

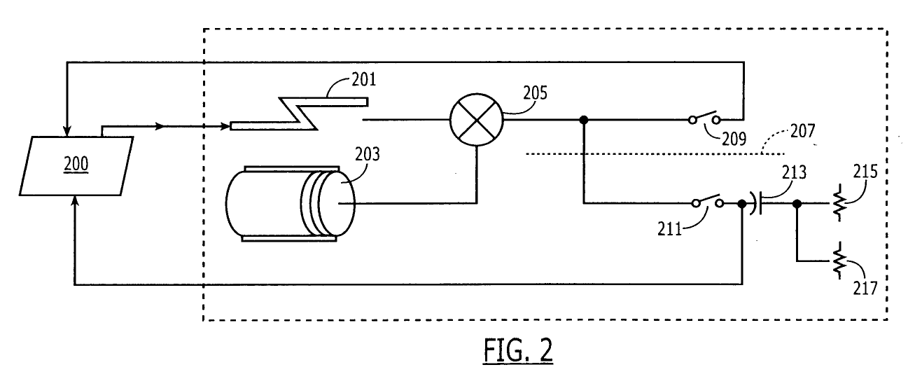

FIG. 2 shows schematic embodiment of how a user's SPO₂ signal is measured and, treatment of sleep apnea, if required.

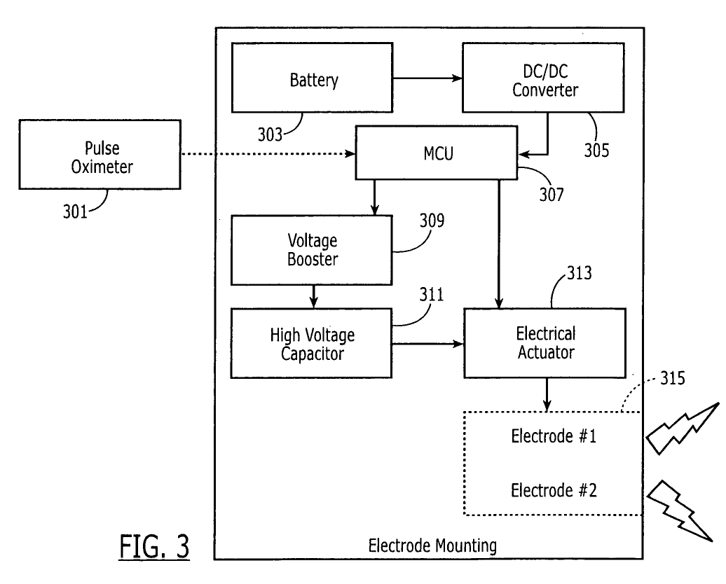

FIG. 3 shows the layout of the device of the present invention.

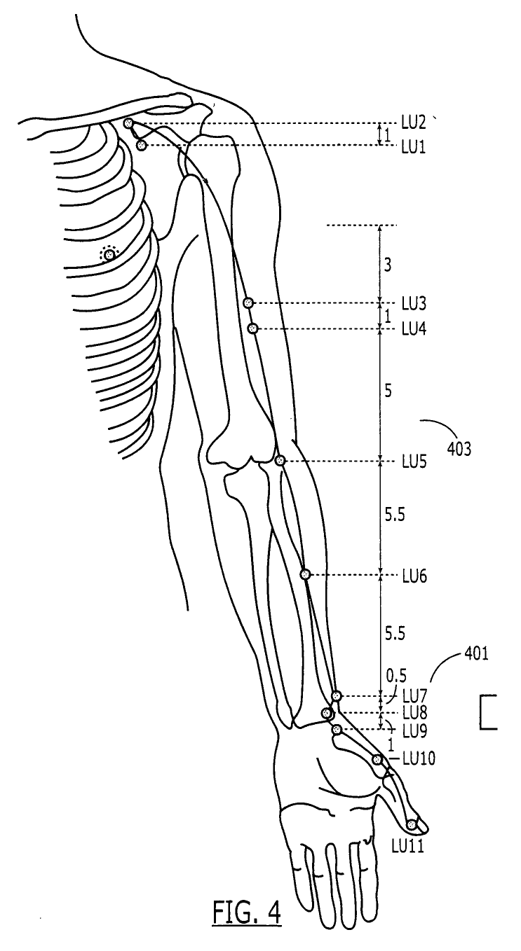

FIG. 4 exhibits the Lung luo-connecting points of the body.

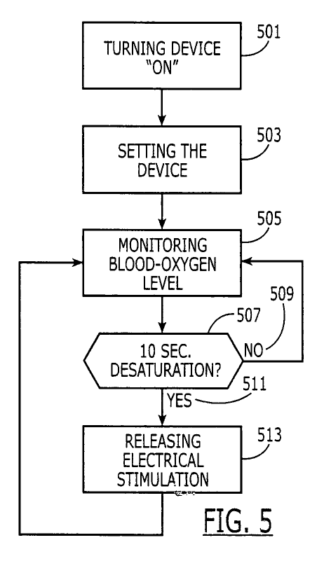

FIG. 5 shows the method of monitoring and treating sleep apnea in accordance with the instant invention.

The following description of certain exemplary embodiment(s) is merely exemplary in nature and is in no way intended to limit the invention, its application, or uses. Throughout this description, the term “SpO₂” shall refer to a measurement of blood-oxygen level.

Now, to FIGS. 1-5,

FIG. 1 is an embodiment of an apnea device 100 in accordance with the present invention. The device 100 is useful for managing apnea during sleep, such management being performed by a pulse oximeter, and stimulation to the user being provided to the Lieque (LU7) meridian point. The device 100 can include a pulse oximeter 101, a connection means 103, an energy source 105, a micro-control unit 107, electronic circuitry 109, smart fabric 111, and electrodes 113.

The pulse oximeter 101 offers a non-invasive measurement of a patient's blood oxygen level. In operation, the oximeter 101 measures absorption at different wavelengths for oxyhemoglobin and its deoxygenated form. The oximeter 101 performs its measurements on preferably translucent parts of the body, such as an earlobe, fingertip, or toe. The oximeter 101 can be positioned on the finger, be handheld, be of tabletop design, wrist-worn, etc. The oximeter 101 includes sensors such as reusable sensors, disposable sensors, finger clip sensors, reflectance sensors, ear clip sensors, etc. The oximeter 101 can contain one or more light emitting diodes, preferably two, with each diode having wavelengths of 660 nm, 905 nm, 910 nm, or 940 nm. The oximeter 101 can further include a connection means 103 to the device. The connection means 103 can be of wireless technology, such as BlueTooth™, infrared, or WIFI. The connection means 103 can be wired technology, can be such as standard pin connectors used in the art, or fiber optic wires. The oximeter 101 may further include digital signal processing. Reflectance probes can alternatively be placed beside the diodes in the oximeter 101.

The device 100 further includes an energy source 105, such as batteries. The device 100 may be powdered by a single AAA size 1.5V. A DC/DC connecter, for example ST5R50M from STMicroelectronics, may be used to set up voltage up to 5V for the whole circuit. Alternatively, the device may be connected to a AC power source, in which case the device 100 can possess necessary AC-power handling components, such as a transformer and/or rectifier.

Through the micro-control unit 107, when the occurrence of sleep apnea is detected by the oximeter 101, the micro-control unit 107 sends a stimulation request signal to the device 100. The stimulation signal initiates the release of electrical stimulation to the LU7 meridian point. In operation, the micro-control unit 107 generates a high-speed pulse width modulation (PWM) signal, which is transmitted to a voltage booster to charge a high voltage capacitor up to a certain voltage level (from 5V to 80V) depending on the chosen electric stimulation level. The electrical actuator, which can have two PNP and two NPN transistors, may be provided to control the energy flow from the high voltage capacitor to the user's skin. The output terminals of the electrical actuator are electrically connected to the conductive electrodes 113.

Electronic circuitry 100 can be suitable for allowing the operation of the device 100. Circuitry 109 can include conductors, resistors, amplifiers, rectifiers, transformers, and the like.

The device 100 includes a smart fiber 111 for attaching to a user's wrist. The attachment on the wrist allows contact between the electrodes 113, and the user's LU7 acupoint. The smart fabric 111 may be made of resilient material, such as plastic, rubber, or composite materials for providing a suitable resilience to wrap around the limb portion of the user. Preferably, the fabric 111 is elastic fabric made of 95% polyester and 5% Lycra™. The elastic fabric may be a plain weft rib knitted surface, knitted by using a tubular knitting machine. The diameter of the fiber may be about 10 microns, and the knitting structure of the fabric may be 16 courses by 20 wales per centimeter.

If desired, fasteners, such as VELCRO-type fasteners, and buckles may be adopted as accessories of the fabric 111. For instance, the fabric 111 may contain two sections, with only one section having two electrically conductive electrodes 113. The section having the two electrically conductive electrodes 113 may be made adjustable in length by using the fastener, and may be connected to the other section of the fabric 111 by the buckles. The fastener may be sewn onto the fabric by lock stitches, and the buckles sewn using hand-stitches.

Two electrically conductive electrodes 113 in the shape of a circular button or disc are embedded inside the fabric 111, with one surface being exposed on the inner surface of the fabric 111. The electrically conductive electrodes 113 may be made of any conductive material, such as metal. In one embodiment, the electrically conductive electrodes 113 are made of 100% stainless steel, or 100% silver coated 100% polyester. The two electrically conductive electrodes 113 can release electrical stimulation upon the skin surface of the user.

Alternatively, the electrically conductive electrodes 113 may be in the form of wires (not shown) woven onto the fabric 111 to form a conductive fabric. The electrically conductive wires may be sewn onto the fabric to make a conductive fabric using an embroidery machine and cotton sewing threads. The width of the embroidery line may be about 2 mm.

To stimulate the user, one of the electrically conductive electrodes 113 has to be located at the target acupoint, such as LU7, and the other electrode 113 may be positioned somewhere else along the Lung meridian.

FIG. 2 is the embodiment of a schematic showing the components of the device that initiates electrical stimulation, such components including memory storage 203, a comparison component 205, switches 209/211, energy storage, components 213, and resistance components such as electrodes 215/217.

Following the micro-control unit 200 sending a stimulation request signal 201 after sleep apnea is detected by a connected oximeter (not shown), the request signal 201 can be sent to the comparison component 205, such as a subtractor. The request signal 201 is a representation of the measurement of blood-oxygen saturation in the user's blood. From the memory storage 203, a reference signal is sent to the comparison component 205 for comparison against the request signal 201. Comparison can occur by subtracting the reference signal from the request signal 201 and obtaining the value thereof. The memory storage 203 can be located on the device, or be located apart thereof. Memory storage 203 can include hard memory, such as ROM, or temporary memory, such as RAM. The information stored on the memory storage 203 includes optimal measurements for the amount of oxygen in the user's blood. The information can be modified to take into account variables such as patient motion, low perfusion, ambient lighting effect, and electrical interference. The information will be in units for comparison against the request signal 201.

The comparison component 205 delivers an error signal following input of the two signals. If the error signal is above a certain level 207, the error signal will actuate one switch 209; if the error signal does not reach the level 207, a second switch 211 will be actuated. In one embodiment, the level is “0” or below; for example if the error signal is “0” or below, the signal will follow the line to the second switch 211. The level at “0” or below would correspond to desaturation in the blood of oxygen. Above “0” would actuate the first switch 209, which will send the signal back toward the micro-control unit 200, in essence issuing an “okay” for the level of oxygen in the user's blood. The actuation of the second switch 211 will lead to the initiation of the energy storage component 213. An example of suitable energy storage component 213 includes capacitors. Energy stored on the energy storage component 213 will not be delivered until a sufficient period of time has passed to satisfactorily indicate the occurrence of sleep apnea. In one embodiment, this period of time is from 10 to 20 seconds. In a preferred embodiment, this time is about 10 seconds. In one embodiment, the energy storage component 213 is connected to a timer, which allows the determination of the passage of time.

Following the passage of sufficient time, a signal can be sent to the resistance components 215/217. The resistance components 215/217 will then release electrical pulses. The resistance component 215/217 can include electrodes. The pulses can be bi-phasic, burst mode and be in square/rectangular shape. The burst rate can be from a range of 1 to 20 bursts per second, preferably about 2 to about 4 bursts per second. The pulse frequency can be about 100 Hz, and have a width of about 0.2 ms. The pulse burst can occur in total for a range of 1 second to 1 minute, preferably about 8 to about 10 seconds.

Measurement and determination will continue to be made as to whether the user is experiencing sleep apnea for a sufficient time while the user is utilizing the device.

FIG. 3 is a system layout embodiment of the device of the present invention, wherein the device includes a pulse oximeter 301, a battery 303, a DC/DC converter 305, a micro-control unit 307 (MCU), a voltage booster 309, a high voltage capacitor 311, an electrical actuator 313, and electrodes 315.

As shown, the pulse oximeter 301 delivers a signal to a MCU 307. The preprocessed signal is interpreted by the MCU 307. Algorithms for sleep apnea detection can be implemented on the MCU 307, which can decide the occurrence of sleep apnea by the user.

A battery 303 provides an energy source which is then stepped-up via DC/DC converter 305. Following the reference signal from the MCU 307 to the electrical actuator 313 and a voltage booster 309, a high voltage capacitor 311 is charged from the voltage-boosted signal. The electrical actuator 313 actuates two electrodes 315 in response to the signal. The electrodes 315 deliver electrical stimulation from energy stored on the capacitor 311.

FIG. 4 shows the LUNG Acupoints L1-L11403. Each lung point has its function in modulating internal body organs and thus achieves the purpose of treating disorders. The acupoint LU7 401, or Lieque, is located 1.5 inches about the wrist, about 0.2-0.3 inches deep, on the inside of the arm. The point is in the depression superior to the styloid process of the radius. LU7 401 is well-known as the principal point on the Lung channel to expel pathogens from the body. It is indicated for the classic signs of an exterior pattern such as chills and fever, headache, aches, and pains in the neck, shoulders and back, etc., as well as acute oedema of the limbs due to impairment by exterior wind of the Lung's function of regulating the water passages and descending fluids to the bladder.

In the present invention, it is the Lung acupoint, specifically LU7, which is applied the electrical stimulation from the electrode, during the occurrence of apnea. While not to be bounded by the theory, it is believed that the LU7 point connects to airway muscle and stimulation of it can immediately improve the condition of airway during sleep apnea.

FIG. 5 is a method of providing electrical stimulation to the LU-7 point of a user experiencing sleep apnea in accordance with the present invention, containing the steps of turning the device “on” 501, setting the device 503, monitoring the blood-oxygen level with the device 505, determining whether there is a 10 sec. desaturation 507, and releasing electrical stimulation to the LU7 point of the user 513, if required.

The device can include button switches, such as “SET” button and “TEST” button, and LEDs, such as red LED, yellow LED, and green LED. The button switches may also be provided to directly connect to the multi-control unit as a user interface. By pressing the “SET” button, a control signal is sent to a voltage booster, and the energy is stored in a high voltage capacitor. The LEDs of the wristband may be used to display an event log. If the “TEST” button is pressed, the LEDs including the set stimulation intensity will be blinking and the electrical stimulation will be released.

The device can be turned “ON” 501 by shifting a power switch. When the device is turned on, an LED can light up. To set the device 503, the “SET” button can be pressed. By continuously pressing the “SET” button, the LEDs can light up in a sequence of green, yellow and red, which represents low, medium, and high stimulation intensity respectively. For example, if the “SET” button is pressed, the green LED will be on to indicate the device is at level 1 (weakest). By pressing the “SET” button again, the yellow LED will be on to indicate level 2. By pressing the “SET” button again, the red LED will be on to indicate level 3. It will return to level 1 if the “SET” button is pressed further. The LEDs will automatically switch off after a particular setting is selected and the device is ready to use.

To experience the level of electrical stimulation, the user may press the “TEST” button. The user should choose the highest, tolerable intensity of electrical stimulation to achieve a maximum, anti-sleep apnea effect. After testing the intensity of the electrical stimulation, the LEDs will be off and the device is in operation and the electrical stimulation level selected is automatically stored.

The blood-oxygen level of the user can be monitored 505 through the pulse oximeter. The oximeter can be connected through a suitable body part of the user, for example finger, earlope, etc. The oximeter continuously monitors the condition of the user while the device is used. The oximeter continuously analyses whether there is about 10 seconds of oxygen desatuaration in the blood of the user. If there is not about 10 seconds of desaturation 509, the signal from the device is continuously looped for further monitoring. If there is at least 10 seconds of desaturation, the signal is delivered to allow the release of electrical stimulation 503 to the LU7 acupoint. The signal is then looped to allow continuous testing.

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

In interpreting the appended claims, it should be understood that:

a) the word “comprising” does not exclude the presence of other elements or acts than those listed in the given claim;

b) the word “a” or “an” preceding an element does not exclude the presence of a plurality of such elements;

c) any reference signs in the claims do not limit their scope;

d) any of the disclosed devices or portions thereof may be combined together or separated into further portions unless specifically stated otherwise; and

e) no specific sequence of acts or steps is intended to be required unless specifically indicated.

1. A sleep apnea device, comprising
a pulse oximeter;
an energy source;
a micro control unit;
a capacitor;
electric circuitry;
a smart fabric;
and a pair of electrodes which are adapted to be disposed on a user's limb for releasing electrical pulses to an acupoint of said user.

2. The sleep apnea device of claim 1, wherein said pulse oximeter is used on translucent part of the body.

3. The sleep apnea device of claim 1, further comprising memory storage.

4. The sleep apnea device of claim 1, wherein said electronic circuitry can be one or more selected from the group consisting of switches, actuators, DC/DC converters, transformers, and rectifiers.

5. The sleep apnea device of claim 1, further comprising a timer connected to said capacitor.

6. The sleep apnea device of claim 1, further comprising algorithms for sleep apnea detection.

7. The sleep apnea device of claim 1, wherein said energy source is a battery.

8. The sleep apnea device of claim 1, wherein said acupoint is an acupoint along the Lung meridian.

9. The sleep apnea device of claim 8, wherein said acupoint is LU7.

10. The sleep apnea device of claim 1, wherein said smart fabric is a resilient material for providing a suitable resilience to wrap around the wrist of the user.

11. A method of monitoring and treating sleep apnea, comprising the steps of
turning “on” a sleep apnea device of claim 1;
setting said device;
monitoring blood oxygen level of the user via a pulse oximeter;
determining if there is desaturation of the user's blood between about 10 to about 20 seconds;
If desaturation is determinated, then performing the following step,
releasing electrical stimulation to an acupoint;
and
continuing to monitor blood-oxygen level of user.

12. The method of claim 11, wherein setting said device comprises pressing a “SET” button on said device to store energy on a high voltage capacitor.

13. The method of claim 11, further comprising the step of testing said device following setting said device.

14. The method of claim 11, wherein monitoring blood-oxygen level of the user comprises connecting the pulse oximeter to a translucent body part.

15. The method of claim 11, wherein determining if there is desaturation of the user's blood occurs for about 10 seconds.

16. The method of claim 11, wherein releasing said electrical stimulation occurs at an acupoint along the Lung meridian.

17. The method of claim 16, wherein releasing said electrical stimulation occurs at the LU7 acupoint.

18. The method of claim 11, wherein releasing said electrical stimulation comprises releasing a bi-phasic burst signal at a range of 1 to 20 bursts per second for a range of 1 second to 1 minute.

19. The method of claim 18, wherein releasing said electrical stimulation comprises releasing a bi-phasic burst signal at a range of 2 to about 4 seconds for a range of about 8 seconds to about 10 seconds.
